(19) United States
(12) Patent Application Publication
(10) Pub. No.: US 2022/0307948 A1
(43) Pub. Date: Sep. 29, 2022

(54) Integrated Sampling Probe, Valve and Vaporiser

(71) Applicant: JDSE Limited, Shrewsbury, Shropshire (GB)

(72) Inventor: Jeremy Knight, Woodseaves, Market Drayton, Shropshire (GB)

(73) Assignee: JDSE Limited, Shrewsbury, Shropshire (GB)

(21) Appl. No.: 17/619,860

(22) PCT Filed: Jun. 9, 2020
(86) PCT No.: PCT/GB2020/051389

§ 371 Date: Dec. 16, 2021

(30) Foreign Application Priority Data
Jun. 17, 2019 (GB) 1908615.6
Jun. 17, 2019 (GB) 1908617.2
Jan. 17, 2020 (GB) 2000734.0

(51) Int. Cl. G01N 1/20; B01B 1/00; F17C 7/04; F17C 9/02; G01N 1/44

(52) U.S. Cl. CPC G01N 1/2035; B01B 1/005; F17C 7/04; F17C 9/02; G01N 1/44; G01N 2001/205

(57) ABSTRACT

An integrated sampling probe, valve and vaporiser (16) for a liquefied natural gas container is provided which comprises a vaporiser body (24) having a vaporisation chamber (66), a fluid inlet (40) in communication with the vaporisation chamber (66), a fluid outlet (58), and a vaporised-fluid flow path extending from the vaporisation chamber (66) to the fluid outlet (58). The fluid inlet (40) is formed as a critical orifice dimensioned to enable vaporisation of fluid passing into the vaporisation chamber (66), and there is a valve member (63) which is drivable to open and close the critical orifice, along with a heating assembly (50) for heating the valve member (63) to enable vaporisation of fluid passing through the critical orifice and into the vaporisation chamber (66). A sampling probe body (18) is also provided extending from the vaporiser body (24), the sampling probe body (18) having a sampling bore (36) which is in fluid communication with the fluid inlet (40).

### CROSS-REFERENCE TO RELATED APPLICATIONS

This application is a national phase entry under 35 U.S.C. § 371 of International Application PCT/GB2020/051389, filed Jun. 9, 2020, which claims priority to GB Patent Application No. 1908615.6, filed Jun. 17, 2019, GB Patent Application No. 1908617.2, filed Jun. 17, 2019, and GB Patent Application No. 2000734.0, filed Jan. 17, 2020, all of which are herein incorporated by reference in their entirety.

### TECHNICAL FIELD

The present invention relates to an integrated sampling probe, valve and vaporiser for a liquefied natural gas pipeline, as well as a system utilising such an integrated sampling probe, valve and vaporiser. A combined sampling probe and vaporiser for vaporising a sample of liquefied natural gas from a container is also provided.

### BACKGROUND

Natural gas, whilst predominantly comprised of methane, is a mixture of different gaseous hydrocarbons, typically with a range of boiling points, as well as small percentages of other compounds. For ease of transport and storage, the natural gas is typically liquefied by being cooled to approximately −168° C. It can then be transported in its liquid state in ships, road tankers, and/or pipes to be used, for instance, as a fuel source for power stations.

It is desirable to monitor the composition of liquefied natural gas, particularly at transfer points, to measure its energy content, calorific value, and other relevant properties. It may also be further desirable to monitor the composition to see that it does not contain unacceptable compounds, such as sulphur or mercury. This monitoring can be achieved by withdrawing a sample of the liquefied natural gas from the pipe, vaporising the sample, and then analysing it.

In order to retrieve an accurate measurement of the liquefied natural gas, instant vaporisation must occur. If slow heating of the sample occurs, the more volatile components of the liquefied natural gas will begin to vaporise causing fractionation. Consequently, the analysed components are not necessarily representative of the liquefied natural gas from the pipe, as the fractionation results in altered concentrations at the point of analysis.

One of the contributing factors associated with premature slow heating of the sample liquid is via the valve which isolates the sampling and analysis system from the process. If the valve is hotter than the liquefied natural gas, then heat will be transmitted into the sample, and fractionation can occur.

In the art, in order to minimise slow heat gain of the sample as it is transferred to the distant vaporiser, a cooling barrier of liquefied natural gas is provided around the isolation valve and the sample tubing. This becomes sacrificial liquefied natural gas, and is therefore vented to waste once it has become heated. This is an undesirable loss of liquefied natural gas

The present invention seeks to provide a sampling probe, valve and vaporiser which are formed as a single unit without causing unnecessary heating to the sampled liquefied natural gas or requiring sacrificial liquefied natural gas.

### SUMMARY

According to a first aspect of the invention, there is provided an integrated sampling probe, valve and vaporiser for a liquefied natural gas container, the integrated sampling probe, valve and vaporiser comprising: a vaporiser body having a vaporisation chamber, a fluid inlet in communication with the vaporisation chamber, a fluid outlet, and a vaporised-fluid flow path extending from the vaporisation chamber to the fluid outlet, the fluid inlet being a critical orifice dimensioned to enable vaporisation of fluid passing into the vaporisation chamber; a valve member which is drivable to open and close the critical orifice; a heating assembly for heating the valve member to enable vaporisation of fluid passing through the critical orifice and into the vaporisation chamber; and a sampling probe body extending from the vaporiser body, the sampling probe body having a sampling bore which is in fluid communication with the fluid inlet.

Integration of the valve into the vaporiser body provides a discrete unitary component which can be readily attached to a main gas pipeline for sampling analysis. The use of the valve member in the vaporiser ensures that liquified natural gas incoming through the fluid inlet is immediately flash vaporised. The risk of fractionation is significantly reduced. By providing a minimal contact area between the fluid inlet and the valve member, thermalisation with the fluid in the sampling bore is minimised despite the heat of the valve member, and therefore no sacrificial liquefied natural gas must be wasted. Furthermore, there will be a fluid flow across the sampling probe body which keeps the sample cooled as it is sampled, further reducing the risk of fractionation. A singular device capable of sampling, isolating and vaporising liquefied natural gas from a main pipeline would also significantly reduce set-up of the sampling apparatus, thereby reducing the interruption to the flow which would be required during the installation.

The heat exchanger provides the bulk of the vaporiser, and is positioned inside the vaporiser body. This allows vaporisation to be conducted immediately after opening of the valve, reducing the risk of slow vaporisation leading to fractionation.

Optionally, the heat exchanger may include at least one heater receiver, the heating means comprising at least one heating element receivably engagable within the or each heater receiver. The or each heating element may be formed as an elongate heater cartridge.

Insertable heaters, such as heater cartridges, allow for the heat exchanger to be heated with minimal cold spots generated, which might otherwise affect the vaporisation process. It also allows for the generation of a sealed unit which can be provided as a single pipe-mountable device.

Preferably, the heat exchanger is formed as an insert which is receivable into an open end of the vaporiser body. Optionally, the heating assembly may be a flanged insert directly connectable to the vaporiser body.

A pluggable insert for a heating assembly may simplify the assembly of the valve, allowing for complex vaporisation channel geometry to be machined into the heating assembly.

In one preferable embodiment, there may further comprise an access port for receiving a drivable element of the valve member. The drivable element may be formed as an elongate valve spindle which extends through the access port. Furthermore, the access port may be formed as a central bore through the heating assembly for receiving the valve spindle therethrough.

The provision of the access port through a bore of the heating assembly ensures that the valve moving member can be installed without necessarily breaching the thermal insulation which separates the vaporiser body and the vaporiser. This mitigates the risk of alternative thermal pathways being formed.

The valve spindle may have a convex tip forming the valve member, or alternatively may have a concave tip forming the valve member or various advantageous geometries.

The vaporiser body may be formed as a flanged pipeline connector. It is preferred that the entire unit be directly mountable to a pipeline, enabling the user to simply bolt on a full sampling array without further adaptation required.

Optionally, the vaporiser body may include a pipeline section engagable with an existing pipeline, the sampling bore extending into the pipeline section. In one preferable embodiment, a longitudinal extent of the sampling probe body in the pipeline section may be at least half of the total length of the sampling probe body, more preferably may be at least three-quarters of the total length of the sampling probe body, and even more preferably may be or be substantially equal to the total length of the sampling probe body. Preferably, the sampling probe body may at least in part form a wall of the pipeline section.

Where a flanged connector for a pipeline is simply not feasible, for example, for pipelines with small diameters, then an alternative solution may be to directly integrate the sampling probe body and/or vaporiser body with a bespoke pipeline section. In this situation, there is no T-junction which would require a baffle or similar deflector plate to urge cool liquefied natural gas flowing through the main pipeline, and as such, the full extent, or majority of the extent, of the sampling probe body can project into the pipeline section.

The fluid flow path may preferably be at least in part formed by a flash vaporisation chamber in the vaporiser body immediately adjacent to the fluid inlet.

Preferably, the heating assembly may comprise a heater which is coupled to the valve member or a drivable element associated therewith. It may be advantageous to associate the heater with the valve member directly, rather than relying on heating through the gap between the heat exchanger and the valve spindle. This will reduce any lag in the heating of the tip of the valve member.

An insulating member positioned inside the vaporiser body will provide a barrier to thermalisation with the walls of the vaporiser body, which could otherwise transmit heat to the fluid inlet.

Optionally, the insulating member may be formed as an insertable sleeve of thermally insulating material. A sleeve may have an advantageous shape, in that it allows for the flanged insert arrangement of the vaporiser to be readily inserted therein and to be cocooned by the sleeve. The protection of the critical orifice from the heat generated by the heating assembly limits the scope for fractionation of the sample.

The vaporiser body may further comprise a sampling probe body extending from the vaporiser body, the sampling probe body having a sampling bore which is in fluid communication with the fluid inlet. Since the use of the vaporiser is linked to sampling liquefied natural gas, it is preferably that the device be provided with the sampling probe body, preferably integrally formed thereon, for ready attachment to a liquefied natural gas pipeline.

Preferably, the sampling probe body may include a baffle thereon for directing fluid flow towards a vaporiser-proximal portion of the sampling probe body. The flow-directing baffle has the advantageous effect of directing very cold liquefied natural gas along and around the sampling probe body. This has a cooling effect on fluid already drawn into the sampling bore, and may further reduce the heating effects which may still be in effect due to the proximity of the vaporiser member.

Optionally, the critical orifice may be formed by an orifice plate received within the vaporiser body.

The provision of a dedicated orifice plate for the fluid inlet allows for simpler machining of the interface between the probe body fluid inlet itself.

According to a second aspect of the invention, there is provided a liquefied natural gas pipeline sampling system comprising: a container for containing liquefied natural gas; and an integrated sampling probe, valve and vaporiser in accordance with the second aspect of the invention, the integrated sampling probe, valve and vaporiser being engagable with the container such that the sampling probe body at least in part extends into the container for sampling liquefied natural gas therein.

Optionally, the container may be a pipeline for transporting flowing liquefied natural gas, the pipeline having a flanged access port, wherein the flanged access port is below a horizontal plane of the main pipe section. A downward configuration may improve gravitational flow of the liquid natural gas into the neck of the flange to cool the sampling probe body

According to a third aspect of the invention, there is provided a combined sampling probe and vaporiser for vaporising a sample of liquefied natural gas from a container, the sampling probe and vaporiser comprising: a vaporiser body having a vaporiser chamber having a vaporisation chamber, fluid inlet in communication with the vaporisation chamber, a fluid outlet, and a vaporised-fluid flow path extending form the vaporisation chamber to the fluid outlet, the fluid inlet being a critical orifice dimensioned to enable vaporisation of a fluid passing into the vaporisation chamber; a vaporiser assembly to enable vaporisation of fluid passing through the fluid inlet and into the vaporisation chamber; and a sampling probe body extending from a container-internal surface of the vaporiser body, the sampling probe body having a sampling bore which is in fluid communication with the fluid inlet; the combined sampling probe and vaporiser being engagable directly or indirectly with the container such that the container-internal surface of the vaporiser body is exposed to a flow of liquefied natural gas through the container.

The exposure of a constant cooling flow of liquefied natural gas across the container-internal surface of the device ensures that there is minimal risk of fractionation of the same before it reaches the critical orifice for vaporisation.

According to a fourth aspect of the invention, there is provided a method of reducing liquefied natural gas wastage during a sampling process from a liquefied natural gas pipeline, the method comprising the steps of: a] providing a thermal barrier between a vaporiser body of an integrated sampling probe, valve and vaporiser attached to a main pipe section of the liquefied natural gas pipeline; b] providing a valve member which is drivable between an open condition and a closed condition of a fluid inlet; and c] heating the valve member such that the valve member acts as a vaporiser for fluid from the fluid inlet in the open condition without the need for sacrificial cooling liquefied natural gas.

According to a fifth aspect of the invention, there is provided a valve body for a sampling port of a liquefied natural gas pipeline, the valve body comprising: a vaporiser body having a fluid inlet and a fluid outlet, a fluid flow path extending through the vaporiser body from the fluid inlet to the fluid outlet; and a heating assembly which is inside the vaporiser body; and an access port for a drivable element of a valve which can close and/or open at least one of the fluid inlet, the fluid outlet, and/or the fluid flow path.

According to a sixth aspect of the invention, there is provided a thermally-controllable valve for a sampling port of a liquefied hydrocarbon sampling container, the valve comprising: a valve body having a vaporisation chamber, a fluid inlet in communication with the vaporisation chamber, a fluid outlet, and a vaporised-fluid flow path extending from the vaporisation chamber to the fluid outlet, the fluid inlet being an orifice dimensioned to enable vaporisation of fluid passing into the vaporisation chamber; a valve member which is drivable to open and close the fluid inlet; and a heating assembly for heating the valve member to enable vaporisation of fluid passing through the fluid inlet and into the vaporisation chamber.

According to a seventh aspect of the invention, there is provided a valve with integrated vaporiser for a sampling port of a liquefied natural gas container, the valve comprising: a valve housing having a valve inlet and a valve outlet, a fluid flow path extending through the valve housing from the valve inlet to the valve outlet; a heating assembly which is inside the valve housing; and a valve member which is drivable between an open condition and a closed condition of the fluid inlet, the valve member being in thermal communication with the heating assembly to form a vaporisation surface for fluid from the valve inlet in the open condition.

According to an eighth aspect of the invention, there is provided a vaporiser for sampling liquefied natural gas that connects directly onto a process line or containment vessel for liquefied natural gas without requiring an external sample line or separate isolation valve.

According to a ninth aspect of the invention, there is provided a vaporiser having an internal means of closing off an inlet flow into a vaporiser body of the vaporiser.

According to a tenth aspect of the invention, there is provided a vaporiser for sampling liquefied natural gas or liquefied petroleum gas that provides heat for overcoming the latent heat of vaporisation that prevents, isolates or minimises the heat from contacting the sample prior to conversion from the liquid state to the gaseous state.

Preferably, the vaporiser may provide additional heat to increase the temperature of the gaseous flow to a required temperature.

According to an eleventh aspect of the invention, there is provided a vaporiser for sampling liquefied natural gas inside of which is a means of isolating the liquid from entering the vaporiser and in which the isolating means is heated.

According to a twelfth aspect of the invention, there is provided a vaporiser with integrated valve for a sampling port of a liquefied natural gas container, the vaporiser comprising: a vaporiser body having a vaporisation chamber, fluid inlet in communication with the vaporisation chamber, a fluid outlet, and a vaporised-fluid flow path extending from the vaporiser chamber to the fluid outlet, the fluid inlet being a critical orifice dimensioned to enable vaporisation of fluid passing into the vaporisation chamber; a valve member which is drivable to open and close the critical orifice; and a heating assembly for heating the valve member to enable vaporisation of fluid passing through the critical orifice and into the vaporisation chamber.

### BRIEF DESCRIPTION OF THE DRAWINGS

The invention will now be more particularly described, by way of example only, with reference to the accompanying drawings, in which:

FIG. 1 shows an end view into a liquefied natural gas pipeline sampling system in accordance with the fourth aspect of the invention, with the interior of the main pipe section being visible;

FIG. 2 is a side representation of the liquefied natural gas pipeline sampling system of FIG. 1, showing a cross-section along line C-C through the main pipe section only;

FIG. 3 shows a full cross-sectional representation of the liquefied natural gas pipeline sampling system of FIG. 1 along line C-C, indicating the valve formed in accordance with the first aspect of the invention;

FIG. 4 shows an enlarged cross-sectional representation of the valve shown in FIG. 3 in box B-B;

FIG. 5 shows an enlarged perspective cross-section through a second embodiment of a valve in accordance with the first aspect of the invention; and

FIG. 6 shows a perspective representation of the liquefied natural gas pipeline sampling system utilising the valve of FIG. 5.

### DETAILED DESCRIPTION

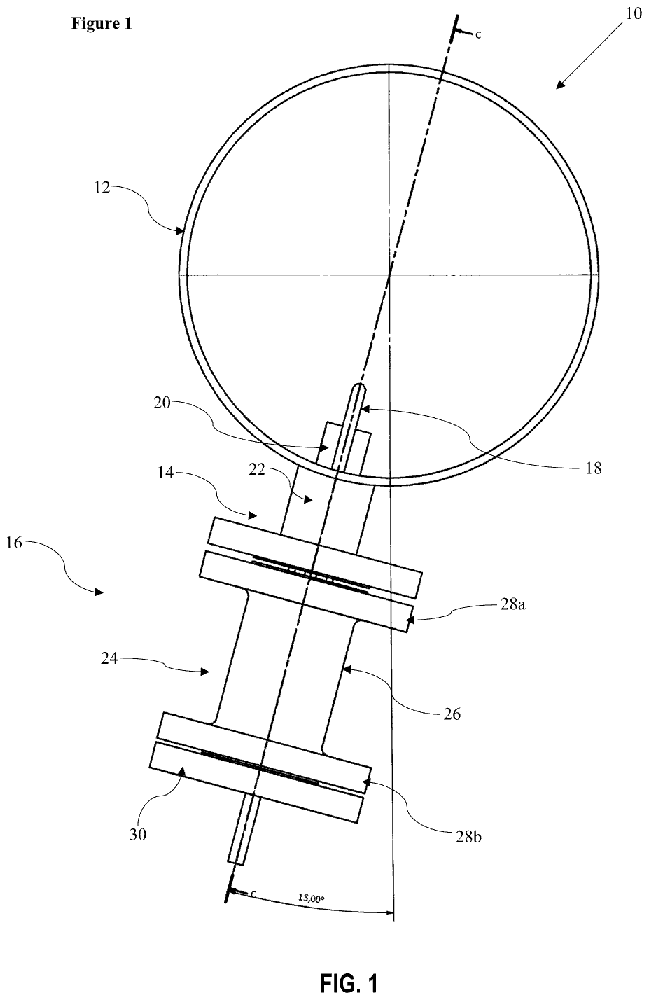

Referring to FIG. 1, there is shown a liquefied natural gas pipeline sampling system, referenced globally at 10, which is suitable for sampling liquefied natural gas flowing through a main pipe section 12. The main pipe section 12 has a flanged access port 14 which extends therefrom, here being offset from a longitudinal vertical plane of the main pipe section 12, for instance, by 15°, and is in a most preferred embodiment, positioned below the horizontal plane of the main pipe section 12.

An integrated sampling probe, valve and vaporiser 16 having an integrated valve is provided which is connectable to the flanged access port 14, which has a sampling probe body 18 which extends into a main flow path of the main pipe body 12, to collect samples of the liquefied natural gas, preferably into a central third thereof for optimum sampling conditions. A baffle 20 is also illustrated, which directs liquefied natural gas flow into the neck 22 of the flanged access port 14.

The integrated sampling probe, valve and vaporiser 16 includes a vaporiser body 24, formed as a tubular pipe section 26 having first and second flanges 28a, 28b at either end thereof, and a flanged insert 30 which is receivable within the tubular pipe section 26.

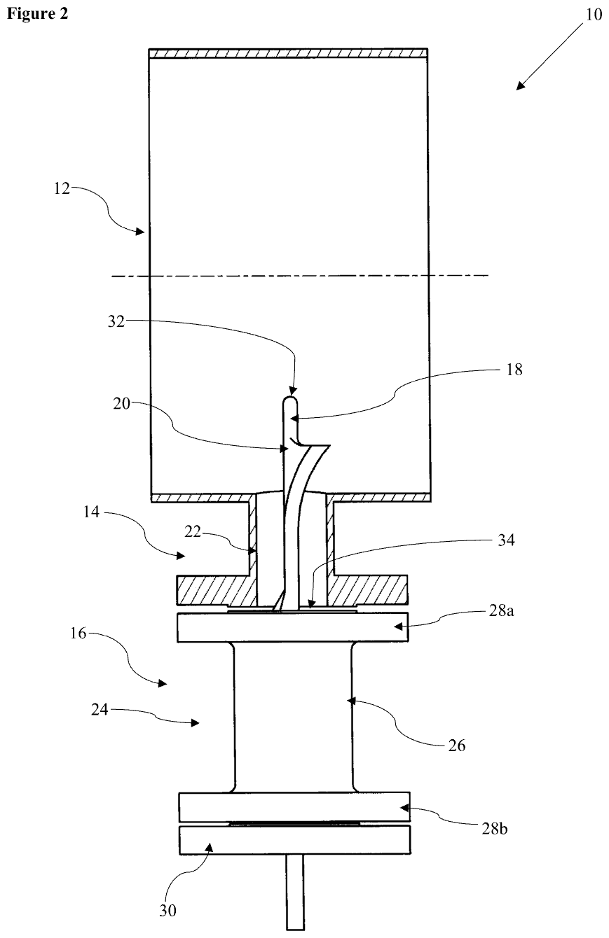

The sampling probe body 18 can be seen in more detail in FIG. 2. The sampling probe body 18 is formed as an elongate rod which is mounted at or adjacent to the first flange 28a of the vaporiser body 24, and has a length which is greater than that of the tubular pipe section 26 such that a sampling tip 32 of the sampling probe body 18 is therefore positioned in the main flow path of the main pipe body 12.

The baffle 20 is arranged to point against the fluid flow, which flows right-to-left in FIG. 2. The baffle 20 has a scoop-shaped end which directs liquefied natural gas into the neck 22 to provide a cooling effect to the sampling probe body 18. At a flange-proximal, or container-internal, end 34 of the sampling probe body 18 and/or vaporiser body 24, there may be one or more channels therethrough via which liquefied natural gas can pass to improve this cooling effect, preferably at or adjacent to the first flange 28a of the vaporiser body 24. This provides for more circulation of the liquefied natural gas in the neck 22.

Other baffle geometries may be provided; to provide the improved cooling effect it is merely required that there is some kind of director or deflection plate which directs the liquefied natural gas flow into the neck 22. The baffle 20 could also be removed completely, if heating of the sampling probe body 18 is not expected to be a major concern.

In particular, it may be that certain arrangements allow for the projecting portion of the sampling probe body 18 to extend into the main pipe section 12 without the need for a flanged access port 14. This may be particularly applicable for small-diameter main pipe sections which would be unable to support a flanged access port. In this instance, liquefied natural gas need not be directed into a neck 22, and a longitudinal extent of the sampling probe body 18 may extend into the pipeline section 12.

The longitudinal extent may be at least half of the total length of the sampling probe body 18, more preferably may be at least three-quarters of the total length of the sampling probe body 18, and even more preferably may be or be substantially equal to the total length of the sampling probe body 18. In one specific embodiment, the sampling probe body 18 may at least in part form a wall of the main pipeline section 12, such that the container-internal surface 34 flushly meets the internal surface of the main pipeline section 12, obviating the need for a baffle 20.

The outer surface of the sampling probe body 18 is preferably smoothed so as to minimise the disruption to the flow of the liquefied natural gas passing thereover. However, in some arrangements, flow diverter elements may be present on the sampling probe body 18, additionally or alternatively 20 to the baffle, to change the flow thereover. For instance, fins or similar elements to minimise forces on the sampling probe body could be considered. Such elements could also provide some of the functionality of the baffle 20.

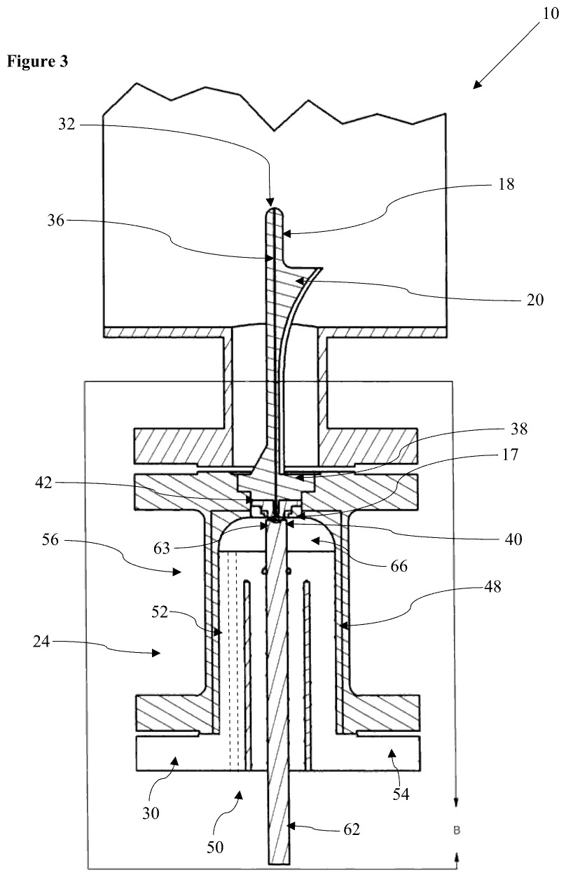

The full cross-section of the integrated sampling probe, valve and vaporiser 16 can be seen in FIG. 3, and a sampling bore 36 of the sampling probe body 18 can also be seen. The sampling bore 36 extends from the sampling tip 32 through the length of the sampling probe body 18, here terminating at a mounting plate 38 of the sampling probe body 18. The mounting plate 38 is directly mounted or mountable to the first flange 28a of the integrated sampling probe, valve and vaporiser 16, preferably by welding or brazing to prevent leak pathways forming into the vaporiser body 24.

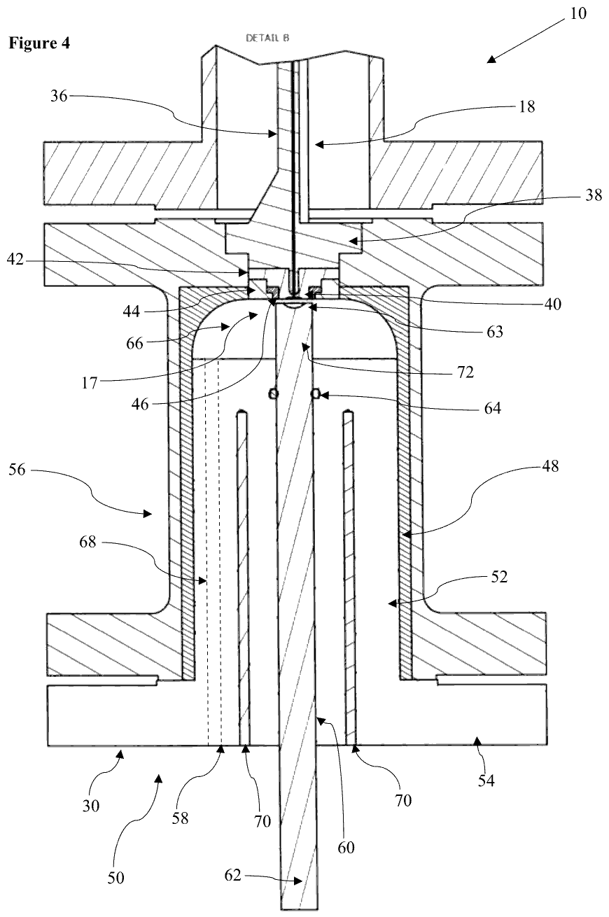

The dimensions of a fluid inlet 40 may be modified by the provision of an orifice plate 42, best illustrated in FIG. 4, which is positionable at or on the mounting plate 38 of the sampling probe body 18. The fluid inlet 40 is preferably formed as a critical orifice, through which flash vaporisation can occur, though non-critical orifices may also work. A critical orifice is an orifice through which critical flow occurs, and is defined as a choke in which the velocity of fluid flow exceeds the velocity of sound in the fluid. This ensures that any disturbance to the fluid stream occurring downstream of the critical orifice cannot be communicated upstream of the critical orifice. Critical flow is preferred, but any appropriate orifice which will result in the fluid being in an appropriate state for vaporisation will be usable within the scope of the present invention.

The fluid inlet 40 may be in contiguous fluid communication with the sampling bore 36, the orifice plate 42 being formed as a top hat structure. A retaining ring 44 may be provided, welded to the first flange 28a, which holds a seal 46 in place on the orifice plate 42. The seal 46 may therefore act as a valve seat, against which a valve member can operate, as will be described hereafter. It will be appreciated that whilst, from a manufacturing perspective, a multi-component construction may be preferred, the first flange 28a, sampling probe body 18, orifice plate 42, seal 46 and/or retaining ring 44 may be integrally formed with one another or in any combination thereof.

It is preferred that, inside the tubular pipe section 26, there is provided an insulating member 48, preferably formed as an insertable sleeve of thermally insulating material, which inhibits thermal transfer between the material of the vaporiser body 24 and the inside of the integrated sampling probe, valve and vaporiser 16. A plastics material such as polytetrafluoroethylene, or silicon glass fabric laminate material could be an appropriate insulating material, such as Tufnol®. A thin-walled insulating member is preferred.

A heating assembly 50 is also provided, which here comprises a heat exchanger 52 and a heating means for heating the heat exchanger 52. The heating assembly 50 is formed as an insert into the vaporiser body 24, such as the flanged insert shown. An insert flange 54 may be directly connectable, for example via welding, to the second flange 28b of the vaporiser body 24 to form a sealed integrated sampling probe, valve and vaporiser 16.

The vaporiser body 24 and heating assembly 50, preferably inclusive of the insulating member 48, collectively form a valve body 56 to control flow through the fluid inlet 40 to a fluid outlet 58 downstream of the fluid inlet 40.

The insulating member 48 may preferably shield the entire heating assembly 50 from the vaporiser body 24; however, it will be appreciated that conduction pathways may be permissible if the rate of conduction does not result in heating upstream of the critical orifice. As shown in the Figures, conduction pathways may be permissible, for example, at or adjacent the insert flange 54, if the rate of heating of the orifice plate 42 is negligible based on the cooling at the proximal end 34 of the sampling probe body 18.

The heat exchanger 52 comprises an access port 60 for receiving a drivable element, such as the valve spindle 62 illustrated, a tip of which acts as the actuating valve member 63, best illustrated in FIG. 4. The access port 60 may preferably be formed as an elongate central bore through the heat exchange 52, having a seal 64 therein for sealingly engaging with the drivable element. An O-ring seal 64 is suitable for a valve spindle 62 drivable element.

The heat exchanger 52 also defines a fluid flow path inside the valve body 56, which allows for vaporised liquefied natural gas to be transported to an analysing station. The fluid flow path in the depicted embodiment comprises a flash vaporisation chamber 66, preferably formed by a void between the internal end of the heat exchanger 52 and the internal surface of the insulating member 48, since the heat exchanger 52 is not dimensioned to fill the entire inner volume of the valve body 56. A path from the flash vaporisation chamber 66 to a vaporiser outlet 58 is formed as a vaporisation channel 68 on the outside of the heat exchanger 52. For manufacturing simplicity, the vaporisation channel 68 is formed on the outer surface of the heat exchanger 52, but it will be appreciated that an internal channel in the heat exchanger 52 would fulfil the same purpose, and may be feasible if the heat exchanger 52 were manufactured using an additive process.

Furthermore, the body of the heat exchanger 52 need not necessarily be cylindrical, but could be polygonal, or similarly geometric. The flow path channel or channels could also be formed by an attachment to a main heat exchanger body, such as by providing fins or ribs on the surface, integrally or not integrally formed.

A heater is provided which is associated with the heat exchanger 52 to form the heating assembly 50. In the depicted embodiment, the heat exchanger 52 has a plurality of elongate receivers 70 which extend into the heat exchanger 52 body, preferably in parallel with the access port 60. Elongate cartridge heaters can then be inserted into the receivers 70 to provide a consistent heating effect therethrough. It will, of course, be appreciated that other heaters could be provided, for instance on the insert flange 54. Since the heat exchanger 52 is formed from a thermally conductive material, the thermal conductance may be sufficient so that cold spots in the vaporiser do not occur.

When taken in conjunction with the valve body 56, the valve member 63 forms a valve for the integrated sampling probe, valve and vaporiser 16. However, the valve member 63 may also act as a flash vaporisation surface within the flash vaporisation chamber 66, since the fluid inlet 40 is directed so as to face the valve member 63.

The valve member 63 here has a concave surface 72, best visualised from the enlarged representation of FIG. 4, which engages with the seal 46 forming the valve seat at the fluid inlet 40. The valve spindle 62 is associated with a drive means, such as a motor, but which could be a manual actuator, to actuate the valve spindle 62 to drive the valve member 63 between closed and open conditions, preferably in a linear motion. Whilst the concave surface 72 is illustrated, it will be appreciated that the sealing surface of the valve member 63 could have any geometry which would appropriately close the fluid inlet 40. This could be concave, as in the present embodiment, convex, as discussed in respect of the second embodiment below, flat, pencil or chisel-tipped, or other shapes which will be apparent to the skilled person.

The operation of the valve is therefore as follows. When the valve spindle 62 is in an open condition, as is shown in FIGS. 3 and 4, liquefied natural gas can flow from the sampling probe body 18, through the sampling bore 36, and into the fluid inlet 40. Liquefied natural gas then passes into the flash vaporisation chamber 66 and directly contacts the hot valve member 63. Flash vaporisation occurs instantaneously, and the risk of fractionation is greatly reduced. The hot valve member 63 therefore acts as the vaporiser, with heat being transferred via the valve spindle 62 via the heat exchanger 52. The vaporised liquefied natural gas can then flow through the vaporisation channel 68 in contact with the heat exchanger 52, so that the sample does not cool and fractionate as it passes towards any analytic equipment downstream of the fluid flow path.

To close the valve, the valve spindle 62 is actuated towards the seal 46 forming the valve seat, closing off the fluid inlet 40. No liquefied natural gas then flows through the fluid inlet 40 and onto the fluid flow path. The valve can then be opened again by linearly actuating the valve spindle 62 away from the fluid inlet 40. The contact between the valve member 63 and the orifice plate 42 or seal 46 is minimal, and is only sufficient to close the fluid inlet 40. As such, thermal conduction from the valve member 63 to the fluid inlet 40 is very small, and therefore the risk of fractionating of the liquefied natural gas sample in the sampling probe body 18 is limited. The presence of the insulating member 48 also serves to isolate the heat exchanger 52 from the orifice plate 42, and therefore additional conduction pathways extending along the vaporiser body 24 to the critical orifice do not form.

The advantages of this arrangement are related to the temperature isolation of the various components. For effective vaporisation, it is not desirable to allow the vaporiser to cool. However, it is very undesirable for thermal transfer through the sampling probe body 18, which could potentially result in alteration of the composition of the sampled liquefied natural gas as some constituents boil too early.

The hot valve member 63 provides a surface against which the liquefied natural gas sample can quickly flash vaporise on entry into the flash vaporisation chamber 66. The hot valve member 63 also has minimal contact with the fluid inlet 40, and therefore thermal conduction pathways are very limited.

The insulating member 48 provides a thermal barrier between the vaporiser body 24 and the heat exchanger 52. This limits the heating of the sampling probe body 18 through the direct connection of the first flange 28a of the vaporiser body 24 to the flanged access port 14 of the main pipe section 12. The void which forms the chamber 66 inside the valve body 56 also provides a thermal break between the heat exchanger 52 and the orifice plate 42 and/or retaining ring 44, so that the fluid inlet 40 does not become heated.

The point of contact via which thermal conduction can occur is where the valve member 63 contacts the seal 46 and/or orifice plate 42. The seal 46 and/or orifice plate 42 itself can be formed from a material having a low thermal conductivity, to mitigate the thermal transfer to the liquefied natural gas sample in the sampling bore 36.

Notwithstanding, the baffle 20 on the sampling probe body 18 further acts to counteract any heating effect produced by the valve spindle 62 being in contact with the orifice plate 42, since cold liquefied natural gas is diverted up the neck 22. This cools the mounting plate 38, which in turn provides a cooling effect to the orifice plate 42 and fluid inlet 40. Of course, in the embodiment where the container-internal surface 34 is at or adjacent to an internal pipe surface of the main pipe section 12, then the natural flow of liquefied natural gas will cool the mounting plate 38.

The orientation of the integrated sampling probe, valve and vaporiser 16 is also important. If, instead of the standard vertical configuration of sampling apparatus as is used in the art, the integrated sampling probe, valve and vaporiser 16 is instead mounted to the main pipe section 12 below a horizontal plane thereof, then there will be many advantages.

Firstly, warmer liquefied natural gas bubbles will not collect in the neck 22 of the flanged access port 14. The warmer bubbles will rise and escape, thereby not imparting a heating effect to the sampling probe 18.

Secondly, the injection of the liquefied natural gas sample into the flash vaporisation chamber 66 against the direction of gravity will create a naturally turbulent flow inside the flash vaporisation chamber 66 and therefore on the fluid flow path. This will encourage mixing of the vaporised sample, and will actively prevent post-vaporisation fractionation prior to analysis.

The method of reducing liquefied natural gas wastage during a sampling process from a liquefied natural gas pipeline can therefore be summarised as follows. A thermal barrier between a vaporiser body 24 of an integrated sampling probe, valve and vaporiser 16 attached to a main pipe section 12 of the liquefied natural gas pipeline. A valve member 63 is provided which is drivable between an open condition and a closed condition of a fluid inlet 40. The valve member 63 is then heated such that the valve member 63 acts as a vaporiser for fluid from the fluid inlet 40 in the open condition without the need for sacrificial cooling liquefied natural gas.

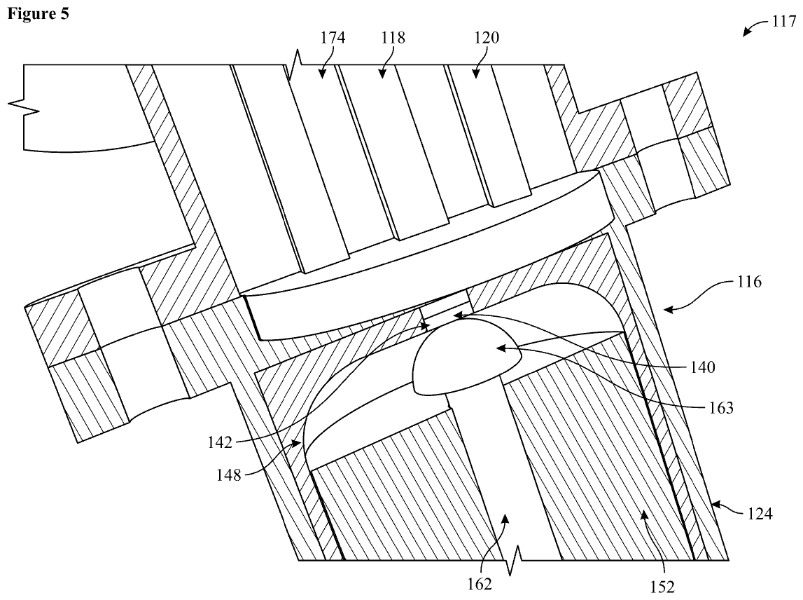

An alternative valve configuration is shown in FIG. 5, the valve being indicated globally at 117. Identical or similar components to those described in respect of the first embodiment of the valve will be referenced using identical or similar reference numerals, and further detailed description will be omitted for brevity.

The integrated sampling probe, valve and vaporiser 116 has a vaporiser body 124, preferably having an insulating member 148 within which is housed the heat exchanger 152. A flash vaporisation chamber 166 is then formed between the heat exchanger 152 and the insulating member 148 at or adjacent to the orifice plate 142.

The sampled liquefied natural gas is drawn through the sampling probe body 118 towards the fluid inlet 140, whilst the baffle 120 directs cooling gas across the base of the sampling probe body 118 to maintain the low temperature at or adjacent to the heated valve member 163. It is noted that the channels 174 through the baffle 120 as previously described are illustrated in respect of the present embodiment.

The valve member 163 here has a convex surface, and therefore the very tip thereof contacts the fluid inlet 140 with a very small contact area. When the valve member 163 is retracted via the valve spindle 162, the fluid inlet 140 is opened, and liquefied natural gas can enter the flash vaporisation chamber 166. Flash vaporisation then can immediately occur on the surface of the valve member 163.

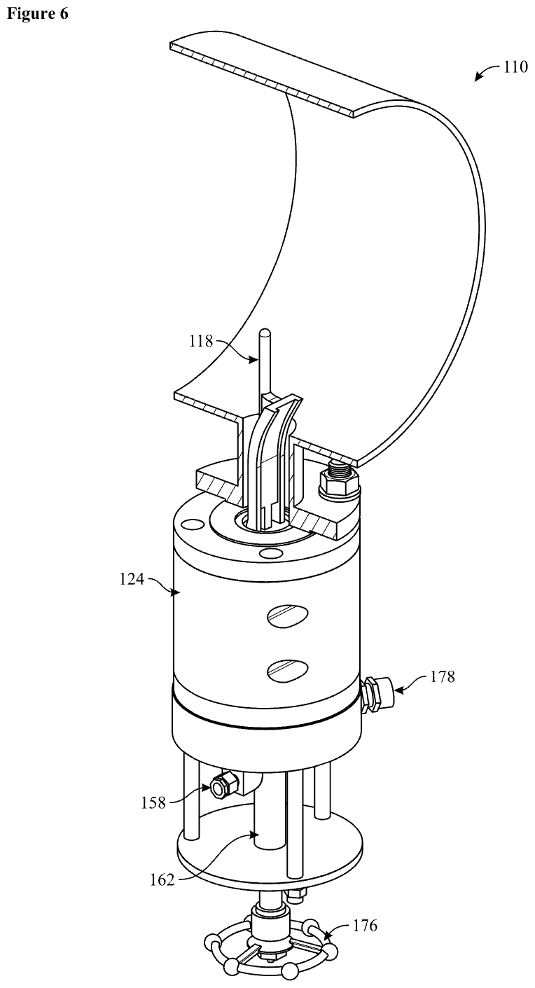

The whole liquefied natural gas pipeline sampling system 110 is illustrated in FIG. 6. The manual drive handle 176 which is connected to the valve spindle 162 can be seen, which allows for a user to manually open or close the valve 117, though this could also be pneumatically, electrically, or hydraulically operated. The external fluid outlet 158 can also be seen, which has a connector, preferably a screw-threaded connector, for engaging with a pipe manifold to an analysis system. One or more electrical couplings 178 may also be provided, which allow for, for instance, connection of a power source to the heating means and/or to one or more temperature sensors which are internal to the vaporiser body 124.

Whilst the present invention is described in respect of the sampling of liquefied natural gas, it will be appreciated that any gas for sampling where it is undesirable to heat the sample prior to vaporisation could find utility within the present invention.

The valve member here, in the form of the spindle valve, directly closes the fluid inlet. However, it will be appreciated that the valve could be adapted to close off any appropriate part of the fluid flow path, from the fluid inlet to the fluid outlet, and would still achieve the same opening and/or closing effect.

Whilst a heat exchanger having a plurality of heating cartridges is herebefore described, it may additionally or alternatively be possible to provide a valve spindle or drivable element which is directly heated, for example, by insertion of a cartridge heater into the drivable element directly. This would allow for direct conduction of heat to the valve member, rather than relying upon heat transfer through the air gap between the heat exchanger and the valve spindle. Said heater could alternatively be positioned directly in the valve member, with electrical wiring passing through the valve spindle.

Whilst the valve arrangement is herebefore described as being mounted to a flanged access port of a pipeline for the measurement of flowing liquefied natural gas, it will be appreciated that the techniques described would be equally applicable in the context of sampling liquefied natural gas from a static storage container, such as a tank or reservoir. No baffle would be required in this scenario, since there is no fluid flow to redirect to towards the probe body.

Additionally, it may be possible, in future container configurations, that a critical or non-critical orifice could be engaged directly onto the outer body of the pipeline. In this scenario, the valve could then be directly mounted to the pipeline, and could even be integrally formed therewith as part of an all-in-one sampling system.

In this case, it may be possible to form a pipeline section which is engagable with an existing pipeline, the sampling bore extending into the pipeline section. The said pipeline section would be integrally formed with, or directly connected to, the vaporiser body, preferably inclusive of an intermediate insulating member. A supplementary gasket or similar seal may be required to maintain fluid tightness. This configuration would be particularly suited to the embodiment described above, in which the container-internal surface of the sampling probe body or vaporiser body is at or adjacent to the internal wall of the pipeline section, preferably so as to be flush therewith.

The arrangement has thus far been described in the context of a vaporiser. However, other heated fluid control equipment could also benefit from the present invention.

For example, it may be desirable for there to be provided a thermally-controlled regulator assembly, particularly in the transport or storage of liquefied petroleum gas. Liquefied petroleum gas does not need to be maintained at such a low temperature as liquefied natural gas in order to remain in the liquid phase. As such, there is less of a burden on the user to maintain a thermal barrier between the sampling probe and any valve member of the regulator. It is still, however, desirable for the valve member to be maintained at a high temperature for the regulator arrangement in order for the liquefied petroleum gas to be raised to temperature rapidly, which again, would potentially result in fractionation of the sample to be analysed inside the regulator body.

The regulator may, instead of a fixed-dimension orifice, have a valve inlet which is dimensionally-adjustable, for instance, based on a pressure differential between regulator chambers. Within the regulator, there may be a diaphragm which is engagable with a valve pin, the orifice changing in dimeter as the pressure changes.

As such, it may be feasible to provide a thermally-controllable valve for a sampling port of a, preferably liquefied hydrocarbon, sampling container, in which the valve member is maintained at a high temperature with respect to the vaporiser body. When the sample is introduced into the valve, its temperature will be rapidly raised, avoiding many of the issues associated with sample fractionation.

The apparatus therefore realises a sampling probe and vaporiser which is provided as a preferably unitary device which can be mounted directly onto a flanged access port of, or otherwise connected to, a main pipe section. This negates the need to provide a separate vaporiser downstream of, in particular, a fluid control valve of the system significantly reduces the assembly complexity of the liquefied natural gas sample vaporisation system.

The words ‘comprises/comprising’ and the words ‘having/including’ when used herein with reference to the present invention are used to specify the presence of stated features, integers, steps or components, but do not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

The embodiments described above are provided by way of examples only, and various other modifications will be apparent to persons skilled in the field without departing from the scope of the invention as defined herein.

1. An integrated sampling probe, valve and vaporiser for a liquefied natural gas container, the integrated sampling probe, valve and vaporiser comprising:
a vaporiser body having a vaporisation chamber, a fluid inlet yin communication with the vaporisation chamber, a fluid outlet, and a vaporised-fluid flow path extending from the vaporisation chamber to the fluid outlet, the fluid inlet being a critical orifice dimensioned to enable vaporisation of fluid passing into the vaporisation chamber;
a valve member which is drivable to open and close the critical orifice;
a heating assembly for heating the valve member to enable vaporisation of fluid passing through the critical orifice and into the vaporisation chamber; and
a sampling probe body extending from the vaporiser body, the sampling probe body having a sampling bore which is in fluid communication with the fluid inlet.

2. The integrated sampling probe, valve and vaporiser as claimed in claim 1, wherein the heating assembly includes a heat exchanger and a heater which is in thermal communication with the heat exchanger.

3. The integrated sampling probe, valve and vaporiser as claimed in claim 2, wherein the heat exchanger includes at least one heater receiver, the heater comprising at least one heating element receivably engagable within the or each heater receiver.

4. The integrated sampling probe, valve and vaporiser as claimed in claim 2, wherein the heat exchanger is formed as an insert which is receivable into an open end of the vaporiser body.

5. The integrated sampling probe, valve and vaporiser as claimed in claim 4, wherein the heat exchanger is a flanged insert directly connectable to the vaporiser body.

6. The integrated sampling probe, valve and vaporiser as claimed in claim 1, further comprising an access port for receiving a drivable element of the valve member (63; 163).

7. The integrated sampling probe, valve and vaporiser as claimed in claim 6, wherein the drivable element is an elongate valve spindle which extends through the access port.

8. The integrated sampling probe, valve and vaporiser as claimed in claim 7, wherein the access port is a central bore through the heating assembly for receiving the valve spindle therethrough.

9. The integrated sampling probe, valve and vaporiser as claimed in claim 7, wherein the valve spindle has a concave or convex tip forming the valve member.

10. (canceled)

11. (canceled)

12. The integrated sampling probe, valve and vaporiser as claimed in claim 1, wherein a longitudinal extent of the sampling probe body in an associated pipeline section is at least half of the total length of the sampling probe body.

13. (canceled)

14. The integrated sampling probe, valve and vaporiser as claimed in claim 12, wherein the said longitudinal extent of the sampling probe body in the pipeline section is or is equal to the total length of the sampling probe body.

15. (canceled)

16. (canceled)

17. The integrated sampling probe, valve and vaporiser as claimed in claim 1, further comprising an insulating member inside the vaporiser body to at least in part thermally isolate the heating assembly from the vaporiser body.

18. The integrated sampling probe, valve and vaporiser as claimed in claim 17, wherein the insulating member is an insertable sleeve of thermally insulating material.

19. The integrated sampling probe, valve and vaporiser as claimed in claim 17, wherein the insulating member provides a barrier between the heating assembly and the vaporiser body at at least the vaporisation chamber.

20. The integrated sampling probe, valve and vaporiser as claimed claim 1, wherein the sampling probe body comprises a baffle thereon for directing fluid flow towards a valve-proximal portion of the sampling probe body.

21. The integrated sampling probe, valve and vaporiser as claimed in claim 1, wherein the critical orifice is an orifice plate received within the vaporiser body.

22. A liquefied natural gas sampling system comprising: a container for containing liquefied natural gas; and an integrated sampling probe, valve and vaporiser as claimed in claim 1, wherein the integrated sampling probe, valve and vaporiser are engagable with the container such that the sampling probe body at least in part extends into the container for sampling liquefied natural gas therein.

23. The liquefied natural gas sampling system as claimed in claim 22, wherein the container is a pipeline for transporting flowing liquefied natural gas, the pipeline having a flanged access port, wherein the flanged access port is below a horizontal plane of the main pipe section.

24. The liquefied natural gas sampling system as claimed in claim 22, wherein the container includes a pipeline section, the sampling probe body being integrally formed with the pipeline section.

25. (canceled)

26. A thermally-controllable valve for a sampling port of a liquefied hydrocarbon sampling container, the valve comprising:
a valve body having a vaporisation chamber;
a fluid inlet in communication with the vaporisation chamber;
a fluid outlet;
a vaporised-fluid flow path extending from the vaporisation chamber to the fluid outlet,
wherein the fluid inlet is an orifice dimensioned to enable vaporisation of fluid passing into the vaporisation chamber;
a valve member which is drivable to open and close the fluid inlet; and
a heating assembly for heating the valve member to enable vaporisation of fluid passing through the fluid inlet and into the vaporisation chamber.
